(19) United States
(12) Patent Application Publication
(10) Pub. No.: US 2014/0010848 A1
(43) Pub. Date: Jan. 9, 2014

(54) GAS-FILLED MICROBUBBLES AND SYSTEMS FOR GAS DELIVERY

(71) Applicants: John Kheir, Boston, MA (US); Francis X. McGowan, Charleston, SC (US); Andrew Loxley, Bethlehem, PA (US); Robert Lee, Bethlehem, PA (US)

(72) Inventors: John Kheir, Boston, MA (US); Francis X. McGowan, Charleston, SC (US); Andrew Loxley, Bethlehem, PA (US); Robert Lee, Bethlehem, PA (US)

(73) Assignee: Children's Medical Center Corporation, Boston, MA (US)

(21) Appl. No.: 13/884,658

(22) PCT Filed: Nov. 11, 2011
(86) PCT No.: PCT/US11/60368

§ 371 Date: Sep. 24, 2013

Related U.S. Application Data
(60) Provisional application No. 61/413,241, filed on Nov. 12, 2010.

(51) Int. Cl. A61K 9/50; A61M 5/19; A61B 5/029; A61M 16/00; A61K 33/00; A01N 1/02

(52) U.S. Cl. CPC A61K 9/5015; A61K 33/00; A01N 1/021; A61B 5/029; A61M 16/00; A61M 5/19
USPC 424/400; 424/613; 435/1.1; 435/2; 600/526; 128/200.24

(57) ABSTRACT

Compressible and concentrated suspensions containing gas-filled microbubbles, uses thereof for delivering gas into a subject in need thereof, and systems for delivering the compressible suspensions. The gas-filled microbubbles each comprise a gas core surrounded by a lipid membrane, which includes (a) one or more lipids, such as 1,2-disteroyl-sn-glycero-3-phosphocholine (DSPC) or dipalmitoylphosphatidylcholine (DPPC), and (b) one or more stabilizing detergents, such as poloxamer 188, Pluronic F108, Pluronic F127, polyoxyethylene (100) stearyl ether, cholesterol, gelatin, polyvinylpyrrolidone (PVP), and sodium deoxycholate (NaDoc).

### RELATED APPLICATION

This application claims priority to U.S. Provisional Application No. 61/413,241, filed on Nov. 12, 2010, the content of which is hereby incorporated by reference in its entirety.

### BACKGROUND OF THE INVENTION

All human cells require a constant oxygen supply to maintain cellular structure and function. When oxygen delivery decreases below Pasteur's point, cells undergo anaerobic respiration. Clinically, this can lead to critical organ dysfunction (e.g., brain and myocardial injury), which could result in death if not rapidly corrected.

Impairments in oxygen supply can occur during airways obstruction, parenchymal lung disease, or impairments in pulmonary blood flow, circulation, blood oxygen content, and oxygen uptake. Brief interruptions in ventilation or pulmonary blood flow can cause profound hypoxemia, leading to organ injury and death in critically ill patients.

Providing even a small amount of oxygen supply may significantly reduce the death rate or the severity of tissue damage in patients suffering from hypoxia. One conventional attempt to restore the oxygen level in a patient is supportive therapy of patient's respiratory system (e.g., mechanical ventilation). This approach is not suitable for patients with lung injury for various reasons. Emergency efforts are another approach to deliver oxygen in a patient. However, they are often inadequate and/or require too long to take effect due to lack of an adequate airway or overwhelming lung injury.

### SUMMARY OF THE INVENTION

The present disclosure relates to gas-filled microbubbles each containing a lipid membrane encapsulating a gas core, compressible suspensions containing such microbubbles, devices for delivering the compressible suspensions into a subject at high infusion rates, methods for delivering gas using the gas-filled microbubbles; and various uses of the gas-filled microbubbles.

In one aspect, disclosed herein is a gas-filled microbubble containing a lipid membrane and a gas core, which is encapsulated by the lipid membrane. This gas-filled microbubble can have a size less than 10 micron in diameter (e.g., 2-6 microns in diameter).

In some embodiments, the lipid membrane includes (a) a lipid, such as 1,2-disteroyl-sn-glycero-3-phosphocholine (DSPC) or dipalmitoylphosphatidylcholine (DPPC), and (b) one or more stabilizing agents, such as poloxamer 188, a poloxamer having a molecular weight lower than that of poloxamer, Pluronic F108, Pluronic F127, polyoxyethylene (100) stearyl ether (also known as Brij® S 100), cholesterol, gelatin, polyvinylpyrrolidone (PVP), and sodium deoxycholate (NaDoc). The gas core can contain oxygen, carbon dioxide, carbon monoxide, nitric oxide, inhalational anesthetic, hydrogen sulfide, or a mixture thereof. In one example, the gas-filled microbubble contains a gas core consisting of oxygen and a lipid membrane formed by (a) DSPC and poloxamer 188, (b) DSPC and polyoxyethylene (100) stearyl ether, (c) DSPC and cholesterol; (d) DSPC, poloxamer 188, and PVP, or (e) DSPC, Pluronic F108, PVP, and cholesterol.

Any of the gas-filled microbubbles described above can be suspended in a solution (e.g., an aqueous solution) to form a microbubble suspension, which is also within the scope of this disclosure. In some embodiments, this suspension is concentrated, e.g., containing at least 60% (e.g., 70%, 80%, or 90%) by volume a gas (e.g., oxygen). In other embodiments, at least 50% (e.g., 60%, 70%, 80%, and 90%) of the microbubbles in the suspension have sizes between 1 to 10 microns (e.g., 2-6 microns in diameters). In yet other embodiments, no more than 8% of the microbubbles in the suspension have sizes greater than 10 micron in diameter. Optionally, 90% of the microbubbles have sizes between 0.5 to 8 microns in diameters.

In another aspect, disclosed herein are delivery systems for administering a compressible suspension containing gas-filled microbubbles, as described above, into a subject (e.g., a human) at a high infusion rate. Uses of these delivery systems can avoid delivering trapped gas into the subject. The term “trapped gas” refer to gas that is neither encapsulated inside a microbubble nor dissolved in a solution.

In one example, the delivery system comprises (i) a first container filled with a concentrated suspension containing gas-filled microbubbles (e.g., any of those described above) at a concentration of at least 70% by volume; (ii) a second container filled with an aqueous solution (e.g., saline); and (iii) a third container having a first port connected to the first container, a second port connected to the second container, a third port for releasing trapped gas, and a fourth port for connecting to a drug delivery device (e.g., a syringe). Optionally, this delivery system further contains a first pump for controlling flow of the suspension from the first container to the third container and a second pump for controlling flow of the aqueous solution from the second container to the third container.

In another example, the delivery system comprises an inner bag filled with a suspension comprising gas-filled microbubbles, such as those described above, and an outer bag surrounding the inner bag. The inner bag has a port for connecting to a drug delivery device (e.g., syringe). This system is configured such that filling of a solution into the space between the inner bag and the outer bag results in flow of the suspension out of the inner bag through the port toward the delivery device.

In still another example, the delivery system comprises at least one drug delivery device for housing a compressible suspension that contains the gas-filled microbubbles described above. The drug delivery device, preferably having a minimal volume of 100 ml, contains (i) a first port connected to a tube, (ii) a second port for releasing trapped gas, and (iii) a pressure unit for applying pressure to the compressible suspension to cause it to exit through the first port at a flow rate of at least 10 mL/minute. The first port has a diameter sufficient to release the compressible suspension into the tube at this flow rate. In some embodiments, the pressure unit is a syringe plunger. In others, it is a pressure valve connected to an external pressure source such as a pump.

Also disclosed herein is a syringe-based gas infusion apparatus comprising: (a) a first chamber at a first end of the apparatus for housing gas or gas-filled microbubbles, (b) a second chamber at a second end of the apparatus for housing an aqueous diluent, (c) a filter plate separating the first chamber and the second chamber, the filter plate including one central hole (optionally the hole may be positioned other than centrally) and a plurality of peripheral holes, in each of which a filter (e.g., a filter paper) resides, (d) a plunger shaft attached to a compressing disc and a plunger disc, which are movable along the axis of the apparatus integrally, and (e) a port at the first end of the apparatus for connecting the first chamber to a delivery device, the port optionally being covered by a cap. In this apparatus, the plunger shaft, the compressing disc, and the plunger disc are configured such that movement of the plunger shaft from the first end toward the second end of the apparatus causes movement of the compressing disc inside the second chamber toward the filter plate, forcing the aqueous diluent to flow from the second chamber into the first chamber. Optionally, pulling the plunger shaft from the second end toward the first end causes movement of the plunger disc inside the first container toward the filter plate.

In some embodiments, the first chamber is filled with gas or gas-filled microbubbles, which can be in dry powder form or in suspension form, and/or the second chamber contains an aqueous diluent, which can be enclosed inside a breakable bag. When necessary, the bag is attached to the compressing disc. The bag breaks when the compressing disc moves toward the filter plate. In other embodiments, the infusion apparatus described herein includes a plunger disc having a size sufficient to seal the central hole via, e.g., screwing into the central hole. The compressing disc, on the other hand, can have a size sufficient to seal the second chamber.

The infusion apparatus described above can be mounted onto a pole (e.g., an IV pole) via a supporting structure to form an infusion system. Preferably, the infusion apparatus in this system can be adjusted vertically, horizontally, or both. In this system, the infusion apparatus can be connected to a pump (e.g., a syringe pump), which can be either installed with or connected to a computer system, and optionally, a syringe adapter affixed to the infusion apparatus. The syringe adapter permits an interface between the apparatus and the pump. Alternatively or in addition, the system further comprises a plunger adapter affixed to the plunger shaft in the infusion apparatus. The plunger adapter is configured for fitting into a plunger depressor of the syringe pumps.

In yet another aspect, disclosed herein is a method of delivering a gas into a subject in need thereof. This method includes administering to the subject by, e.g., intravenous or intraarterial injection, an effective amount of compressible suspension containing any of the gas-filled microbubbles described above. Preferably, the suspension can have a low viscosity such that trapped gas moves freely within the suspension and therefore are easily excluded from the suspension (i.e., free of trapped gas). In one example, the administering step is performed using a multi-syringe pump. In another example, it is performed using any of the drug delivery systems described above. When necessary, the delivery system is placed in a position (e.g., vertical) to allow release of trapped gas or avoid flow of trapped gas to the delivery device in the system, thereby preventing delivery of trapped gas into the subject.

In some embodiments, oxygen is delivered into a subject in need thereof by the just-described delivery method, using oxygen-filled microbubbles. The infusion rate of a suspension containing oxygen-filled microbubbles can range from 10 to 400 ml/minute of oxygen. The subject in need thereof can be a human patient who is or is suspected of experiencing local or systemic hypoxia. The subject can also be a human patient having or suspected of having congenital physical or physiologic disease, transient ischemic attack, stroke, acute trauma, cardiac arrest, exposure to a toxic agent (e.g., carbon monoxide or cyanide), heart disease, hemorrhagic shock, pulmonary disease, acute respiratory distress syndrome, infection, and multi-organ dysfunction syndrome.

Also disclosed herein are:

A method including administering to a subject in need thereof (e.g., a prematurely born human infant, a human infant suffering from or suspected of having necrotizing enterocolitis, or a human patient suffering from or suspected of having chronic obstructive pulmonary disease) at a site in the abdominal cavity (e.g., the intestine or the peritoneum) or in the thoracic cavity (e.g., pleura) an effective amount of a suspension containing oxygen-filled microbubbles as described above.

A method for organ preservation, including delivering an effective amount of a suspension containing the oxygen-filled microbubbles described above, and optionally, red blood cells, into a blood vessel in an organ (e.g., lung, heart, kidney, liver, skin, cornea, or extremity), which can be an organ to be used in transplantation.

A method for prolonging storage of blood in vitro, including mixing oxygen-filled microbubbles as described above with a blood sample. In some embodiments, the mixing step is repeated periodically during storage of the blood sample.

A method for determining cardiac output noninvasively, by injecting a known amount of oxygen into the venous bloodstream and measuring the time to a change in expired oxygen content or a change in arterial oxygen saturations.

A method for promoting wound healing, including administering (e.g., topically) an effective amount of a suspension containing the oxygen-filled microbubbles described above to a wound site or a site nearby a wound.

A composition formulated for topical administration, comprising any of the gas-filled microbubbles described herein and a topical carrier.

A method for reducing a side effect caused by cancer radio therapy, including administering an effective amount of a suspension containing the oxygen-filled microbubbles described above to a tumor site or a site nearby a tumor in a subject (e.g., a human cancer patient) who has undergone radio therapy.

A method for ameliorating sickle cell crisis, including administering to a subject in need thereof (e.g., a human patient suffering or suspected of having sickle cell anemia) an effective amount of a suspension containing the oxygen-filled microbubbles described above. In one example, the subject has or is suspected of having acute chest syndrome or a vaso-occclusive crisis.

Also within the scope of this disclosure are (a) pharmaceutical suspensions containing any of the gas-filled microbubbles described herein for use in delivery of a gas into a subject in need thereof (e.g., those described herein) and/or treating any of the diseases noted herein (e.g., reducing a side effect caused by cancer radio therapy or ameliorating sickle cell crisis), and (b) uses of the suspensions/gas-filled microbubbles in manufacturing a medicament for use in gas delivery in a subject in need thereof.

The details of one or more embodiments of the invention are set forth in the description below. Other features or advantages of the present invention will be apparent from the following drawings and detailed description of several examples, and also from the appended claims.

### BRIEF DESCRIPTION OF THE DRAWINGS

The drawings are first described.

FIG. 1 is a schematic diagram illustrating a gas-delivering system.

FIG. 2 is a schematic diagram illustrating another gas-delivering system.

FIG. 3 is a photo showing a gas-delivery system containing six syringes connected to a pump.

FIG. 4 is a schematic diagram illustrating a syringe-based gas infusion system. 4A: a diagram showing a syringe-based infusion apparatus for delivering gas or gas-filled microbubbles to a subject. 4B is a diagram showing the filter plate in the apparatus depicted in 4A. The left panel is a top view of the filter plate and the right panel is a front view of the filter plate. 4C is a diagram showing an infusion system containing the infusion apparatus depicted in 4A.

FIG. 5 is a diagram showing particle size distributions of oxygen-filled microbubbles. Panels A and B: percentages of various particles having sizes greater than 10 micron over time.

FIG. 6 is a diagram showing stability of various oxygen-filled microbubbles. A: a chart showing the percentages of remaining microbubbles having membranes formed by DPPC and PEG 40S or BRIJ 100 over time at 4° C. B: a chart showing the percentages of remaining microbubbles having membranes formed by DSPC and PEG 40S, BRIJ 100, poloxamer 188, or DSPE-PEG 2000 over time at 4° C. C: a chart of the percentages of various microbubbles having a size greater than 10 micron over time at 4° C.

FIG. 7 is a bar graph showing increased oxygen saturation in rabbits subjected to infusion of oxygen-filled microbubbles as compared to control rabbits.

FIG. 8 is a diagram showing therapeutic effects of oxygen-filled microbubbles in asphxial rabbits. Panel A: a chart showing real time PaO₂ levels in asphyxial rabbits treated with oxygen-filled microbubbles containing poloxamer 2 (poloxamer 188) and in control rabbits. Panel B: a chart showing PaO₂ levels in asphyxial rabbits treated with oxygen-filled microbubbles and in controls. Panel C: a chart showing mean arterial pressures in oxygen-filled microbubble-treated rabbits and in controls at various time points after asphyxia infusion. Panel D: a chart showing the percent of spontaneous circulation (i.e., percent not requiring CPR) during asphyxia in rabbits treated with oxygen-filled microbubbles and in rabbits treated with oxygenated crystalloid.

### DETAILED DESCRIPTION OF THE INVENTION

The present invention is based at least in part on an unexpected discovery that administering to asphyxial subjects a concentrated suspension containing oxygen-filled microbubbles via intravenous injection successfully restores oxygen supply in the subject, preserves spontaneous circulation during asphyxia, and reduces occurrence of cardiac arrest.

Accordingly, disclosed herein are gas-filled microbubbles each including a lipid membrane encapsulating a gas core, a compressible and concentrated suspension containing such gas-filled microbubbles, systems and methods for delivering compressible suspensions containing gas-filled microbubbles at a high infusion rate, and uses of the compressible suspensions to effectively deliver gas to a subject in need thereof.

### (I) Gas-Filled Microbubbles and Suspensions Containing Such

The gas-filled microbubbles described herein each contain a gas core surrounded by a lipid membrane, which can be either a monolayer or a bilayer. The lipid membrane can contain one or more lipids and one or more stabilizing agents. In some embodiments, the molar ratio of lipid:stabilizing agent ranges from 10,000,000:1 to 1:1, preferably 1,000:1 to 10:1.

A variety of lipids, either naturally-occurring or synthetic, can be used to prepare the lipid membrane of the microbubbles. Typically, the lipids are amphipathic, i.e., comprising a hydrophilic moiety and a hydrophobic moiety. Lipids suitable for making lipid membranes are well known in the art, including, but are not limited to, fatty acids, triacyl glycerol, terpenes, waxes, sphingolipids, and phospholipids (e.g., phosphocholines, phosphoglycerols, phosphatidic acids, phosphoethanolamines, and phosphoserines). See also US 2009/0191244, U.S. Pat. No. 7,105,151, and U.S. Pat. No. 6,315,981, all of which are incorporated herein by reference in their entity. Examples are cholesterol, egg lecithin, Disteroylphosphatidylcholine (DSPC), dimyristoylphosphatidylcholine (DMPC), Dipalmitoylphosphatidylcholine (DPPC), and dioleoyl-sn-glycero-3-phosphocholine (DOPC). In some embodiments, the lipids used for making the gas-filled microbubbles have one or more acyl chains with a length ranging from C₁₂ to C₂₄ (e.g., C₁₆ or C₁₈). Optionally, the acyl chains are saturated.

The term “stabilizing agent” used herein refers to a compound capable of stabilizing the microbubbles by reducing coalescence and/or altering surface tension. Typically, a stabilizing agent contains a hydrophobic moiety, which incorporates into the phospholipid layer, and a hydrophilic component, which interacts with the aqueous phase and minimizes the energy of the microbubble, thereby enabling its stability. Suitable stabilizing agents include detergents, wetting agents, and emulsifiers, all of which are well known in the art. See, e.g., US 2009/0191244, U.S. Pat. No. 7,105,151, and U.S. Pat. No. 6,315,981. Examples include, but are not limited to, poloxamers, polyethylene glycol, nonionic polyoxyethylene surfactant, mannitol, cholesterol, and lecithin.

In some embodiments, the stabilizing agent is a poloxamer such as poloxamer 188 (chemical name Pluronic F68), poloxamer 338 (chemical name Pluronic F108), or poloxamer 407 (chemical name Pluronic F127). Poloxamers are nonionic triblock copolymers composed of a central hydrophobic chain of polyoxypropylene (also known as poly(propylene oxide)) flanked by two hydrophilic chains of polyoxyethylene (also known as poly(ethylene oxide)). The three digit number 188 indicates the approximate molecular mass of the polyoxypropylene core (i.e., 1800 g/mol) and the polyoxyethylene content (i.e., 80%). Poloxamer surfactants are commercially available, e.g., provided by BASF Corporation.

In other embodiments, the stabilizing agent is polyoxyethylene (100) stearyl ether (Brij® S 100), which is commercially available from, e.g., Sigma-Aldrich. In yet other embodiments, the stabilizing agent is cholesterol, NaDOC, gelatin, or PVP.

When necessary, a combination of various stabilizing agents can be used for preparing the gas-filled microbubbles. Such a combination can contain a poloxamer (e.g., poloxamer 188, poloxamer 338, and/or poloxamer 407) and one or more of cholesterol, NaDOC, gelatin, and PVP. Alternatively, the stabilizing agent combination described herein can contain PVP and NaDOC, and/or gelatin. Examples of the combinations include, but are not limited to, those listed in the table in Example 3 below. The concentration of each of the various stabilizing agents can vary and optional concentrations can be determined via routine methodology.

The gas-filled microbubbles described herein can each contain a lipid membrane composed of DSPC and one or more stabilizing agents described herein (e.g., cholesterol). In some examples, the lipid membrane is composed of DSPC and one of the stabilizing agent combinations described above. In other examples, the lipid membrane is composed of one or more lipids (e.g., DSPC, DMPC, DPPC, and/or DOPC) and cholesterol, and optionally one or more other stabilizing agents such as those described herein.

The gas core encapsulated by the lipid membrane described above can contain one or more pharmaceutically acceptable gases, e.g., oxygen, nitrogen, carbon dioxide, carbon monoxide, nitric oxide, helium, argon, xenon, inhalational anesthetic (e.g., isoflurane, desflurane, nitrous oxide, or sevoflorane), or hydrogen sulfide. Typically, the gas core consists of free, unbound gas. When the gas contains oxygen, the microbubbles are free of agents that increase the solubility of the oxygen, such as perfluorocarbon-based liquids, fluorinated gas, or hemoglobin/hemoglobin-based molecules.

The gas-filled microbubbles described herein can be prepared by any conventional methods, including shear homogenization (see Dressaire et al., Science 320(5880):1198-1201, 2008), sonication (see Suslick et al., Philosophical Transactions of the Royal Society of London Series a—Mathematical Physical and Engineering Sciences 357(1751):335-353, 1999; Unger et al., Investigative Radiology, 33(12):886-892, 1998; and Zhao et al., Ultrasound in Medicine and Biology, 31(9):1237-1243, 2005), or extrusion (see Meure et al., AAPS PharmSciTech, 9(3):798-809, 2008), followed by spraying (see Pancholi et al., J. Drug Target. 16(6):494-501, 2008), mixing (see Kaya et al., Ultrasound in Medicine and Biology. 35(10):1748-1755, 2009), or floatation (see Feshitan et al., J. Colloid Interface Sci. 329(2):316-324, 2009) to obtain microbubbles having particle sizes suitable for intravenous uses (i.e., below 10 microns in diameter), and those described in Meure et al., AAPS PharmSciTech 9(3):798-809, 2008.

Typically, a process for preparing gas-filled microbubbles includes at least two steps: (i) mixing lipid(s) and stabilizing agent(s) as described above in a suitable solvent (e.g., an organic solvent or an aqueous solution) to form a pre-suspension, and (ii) dispersing one or more gases into the pre-suspension to form gas-filled microbubbles via, e.g., adsorption of the lipid component to the gas/lipid interface of entrained gas bodies. See, e.g., U.S. Pat. No. 7,105,151. Step (ii) can be performed under high energy conditions, e.g., intense shaking or sonication. See, e.g., US 2009/0191244 and Swanson et al., Langmuir, 26(20):15726-15729, 2010. The microbubbles thus produced, suspended in the solvent used in step (i), can be concentrated and/or subjected to size selection by methods known in the art, such as differential centrifugation as described in US 2009/0191244 to produce highly concentrated suspensions of microbubbles. In some embodiments, the gas content in a concentrated suspension is at least 60% (e.g., 70%, 80%, or 90%) by volume. Alternatively or in addition, the size of the microbubbles is below 10 microns in diameter (e.g., 5-10 microns in diameter, 2-5 microns in diameter, or less than 2 microns in diameter). The size of these microbubbles can be further determined using a suitable device, e.g., Accusizer® or Multisizer® III. Microscopy can be applied to directly visualize the microbubbles in the concentrated suspension.

After the gas-filled microbubbles are delivered into a subject, the gas core reaches an equilibrium across the lipid membrane between the gas core and the surrounding plasma, which may include desaturated hemoglobin. When the gas core contains oxygen, it binds rapidly to hemoglobin, which provides an ‘oxygen sink. This strongly favors a tendency of oxygen to leave the particle's core rather than remain within it. Depending upon the need of a subject, the microbubbles can be designed such that they release the gas or gas mixture immediately following administration (e.g., <10 milliseconds to 1 minute); alternatively, they can be designed to persist in vivo until they reach hypoxic tissue, where the lipid membrane collapses to release the gas or gas mixture.

The gas-filled microbubble suspension described above can be mixed with one or more additional components, such as a pharmaceutically acceptable carrier or excipient (e.g., saline) or another therapeutically active agent. A pharmaceutically acceptable carrier is compatible with the gas-filled microbubbles (and preferably, capable of stabilizing it) and not deleterious to the subject to be treated. Preferably, the suspension contains as little lipid as possible and is isotonic with blood. In one example, the only lipid components in the suspension are those from the lipid membranes of the microbubbles. When necessary, the suspension contains an isotonic agent (e.g., Plasmalyte, 0.9% NaCl, 2.6% glycerol solution, lactated Ringer's solution, and 5% dextrose solution), a volume expander (e.g., Hextend®, hetastarch, albumin, 6% Hydroxyethyl Starch in 0.9% Sodium Chloride Infusion (Voluven®), a blood (e.g. packed red blood cells) or hemoglobin-based oxygen carrier, and/or a physiologic buffer (e.g. tris(hydroxymethyl)aminomethane, “THAM”). Such embodiments are particularly useful in a clinical situation of impaired ventilation. In other embodiments, the suspension can contain one or more cryoprotectants, e.g., glycols such as ethylene glycol, propylene glycol, and glycerol.

In one example, the gas-filled microbubble suspension described above can be formulated in a manner suitable for topical administration, e.g., as a liquid and semi-liquid preparation that can be absorbed by the skin. Examples of a liquid and semi-liquid preparation include, but are not limited to, topical solutions, liniments, lotions, creams, ointments, pastes, gels, and emugels.

In another example, the microbubble suspension is co-formulated with one or more additional therapeutic agents for co-delivery of the gas or gas mixture inside the microbubbles and the one or more agents, which can be, but are not limited to, lipid-soluble drugs, nucleotide acid-based drugs such as siRNAs or microRNAs, protein drugs such as antibodies, or free radical scavengers.

Any of the microbubble-containing compositions described herein can be either in suspension form or in dry powder form (obtained, via spray drying). When in dry powder form, the composition can be mixed with a solution such as saline immediately before use.

The gas-filled microbubble suspensions described above can be used for gas delivery shortly after their preparation. If needed, they can be stored under suitable conditions (e.g., refrigerated conditions) before administration. As shown in Example 1 below, the gas-filled microbubble suspension described herein is very stable under standard refrigerated conditions or at room temperature. An “effective amount” is the amount of the suspension that alone, or together with one or more additional therapeutic agents, produces the desired response, e.g. increase in the local or systemic level of a desired gas such as oxygen in a subject. In the case of treating a particular disease or condition described below, the desired response can be inhibiting the progression of the disease/condition. This may involve only slowing the progression of the disease/condition temporarily, although more preferably, it involves halting the progression of the disease/condition permanently. This can be monitored by routine methods. The desired response to treatment of the disease or condition also can be delaying the onset or even reducing the risk of the onset of the disease or condition. An effective amount will depend, of course, on the particular disease/condition being treated, the severity of the disease/condition, the individual patient parameters including age, physical condition, size, gender and weight, the duration of the treatment, the nature of concurrent therapy (if any), the specific route of administration and like factors within the knowledge and expertise of a health practitioner. These factors are well known to those of ordinary skill in the art and can be addressed with no more than routine experimentation. It is generally preferred that a maximum dose of the suspension be used, that is, the highest safe dose according to sound medical judgment.

### (II) Systems and Methods for Delivering Gas Using Compressible Suspensions Containing Gas-Filled Microbubbles

An effective amount of a compressible suspension containing gas-filled microbubbles as described above can be administered via, e.g., a syringe, to a subject in need, either locally (e.g., via topical administration) or systemically (e.g., via intravenous or intraarterial injection), to deliver a gas or a gas mixture into the subject.

The gas such as oxygen contained in a suspension of microbubbles is either encapsulated inside the microbubbles in gaseous form or dissolved in the liquid phase of the suspension. When gas-filled microbubbles breakdown prematurely (during manufacture, manipulation or storage), the gaseous phase can be released into the suspension. The released gas easily coalesces to form larger collections. If such collections become trapped within the suspension (i.e., trapped gas) to be infused, substantial injury or death to the recipient would occur by way of a gas embolus.

One advantage of the gas-filled microbubble suspension described herein is that it can be free of trapped gas so as to ensure that large collections of trapped gases are not infused into a patient. This can be achieved by adjusting the viscosity of the compressible suspension to a low level (e.g., the suspension is free-flowing or almost free-flowing) such that trapped gas, once formed, escapes from the suspension. The viscosity of a microbubble-containing suspension can be adjusted via conventional methods, e.g., dilution with a crystalloid.

The suspension is delivered into a subject at a suitable flow rate depending upon the subject's need. For example, when the subject needs oxygen supply, a suspension containing oxygen-filled microbubbles can be delivered to that subject at a flow rate of 10 mL/min to 400 mL/min (e.g., 50-300 mL/min or 100-200 mL/min). The flow-rate can also be adjusted based on the subject's oxygen consumption, oxygen saturation, skin and mucous membrane color, age, sex, weight, oxygen or carbon dioxide tension, blood pressure, systemic venous return, pulmonary vascular resistance, and/or physical conditions of the patient to be treated.

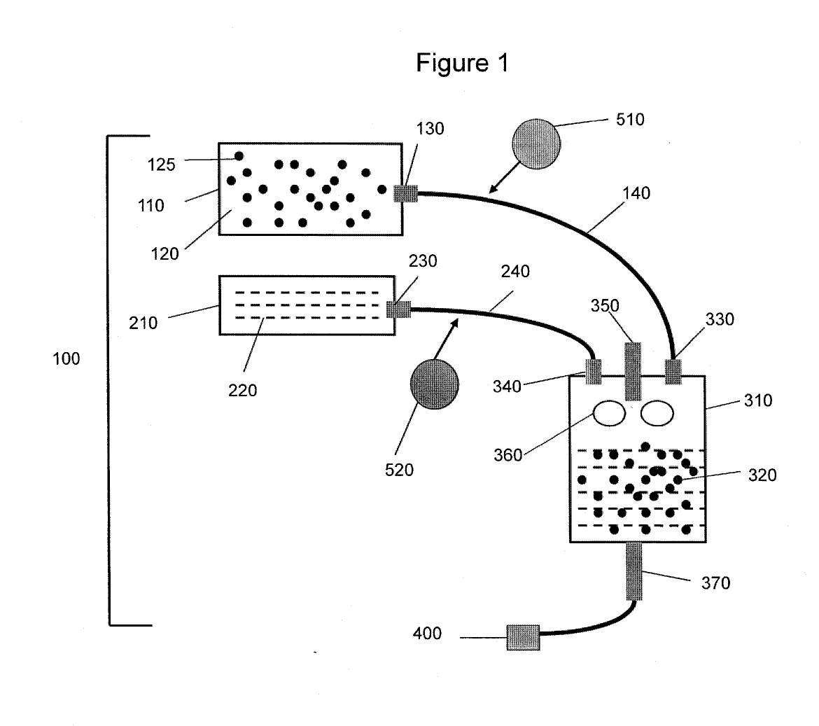

In some embodiments, delivery system 100 depicted in FIG. 1 is used for administrating a compressible suspension containing gas-filled microbubbles described herein. Referring to FIG. 1, this delivery system includes at least three containers, i.e., container 110, container 210, and container 310. Each of container 110 and container 210 has port 130 and port 230, respectively, for connecting to container 310 via port 330 and port 340. In one example, port 130 and port 330 are connected by tube 140 and port 230 and port 340 are connected by tube 240. Concentrated suspension 120 containing gas-filled microbubbles 125 can be placed in container 110, which can flow into container 310 through tube 140. Container 210 can be filled with solution 220 (e.g., an aqueous solution), which can flow into container 310 through tube 240. The flow rates of suspension 120 and solution 220 from container 110/210 to container 310 can be controlled by, e.g., pump 510 and 520, respectively, such that their mixture formed in container 310, i.e., compressible suspension 320, is suitable for administration, e.g., having a suitable gas concentration and a suitable velocity. Each of tubes 140 and 240 has a diameter sufficient to release concentrated suspension 120 or solution 220 at the controlled flow rate.

Container 310 further includes port 350 for releasing trapped gas produced during mixture of suspension 120 and solution 220, and port 370 for connecting to at least one delivery device 400, e.g., a syringe. When necessary, this container is placed during administration in a position (e.g., vertical), at which trapped gas escapes from suspension 320 and releases through port 350.

In addition, Container 310 can further include a pressure unit for applying pressure to compressible suspension 320 to cause it exit from portion 370 to delivery device 400 and subsequently, deliver into a subject who needs the treatment. The pressure unit can be a syringe plunger or a pressure valve connected to an external pressure source (e.g., a pump).

Alternatively, delivery device 400 includes one or more containers for housing compressible suspension 320, a first port connected to a tube, a second port for releasing trapped gas, and a pressure unit as described above. The tube has a diameter sufficient to release suspension 320 into the tube at a suitable flow rate (e.g., 200 mL/minute), and subsequently, to a subject in need of the treatment. Preferably, the container(s) for housing the compressible suspension has a minimal volume of 500 mL. In one example, the delivery device is a multi-syringe pump, e.g., the NE-1600 multi-syringe pump provided by New Era Pump Systems.

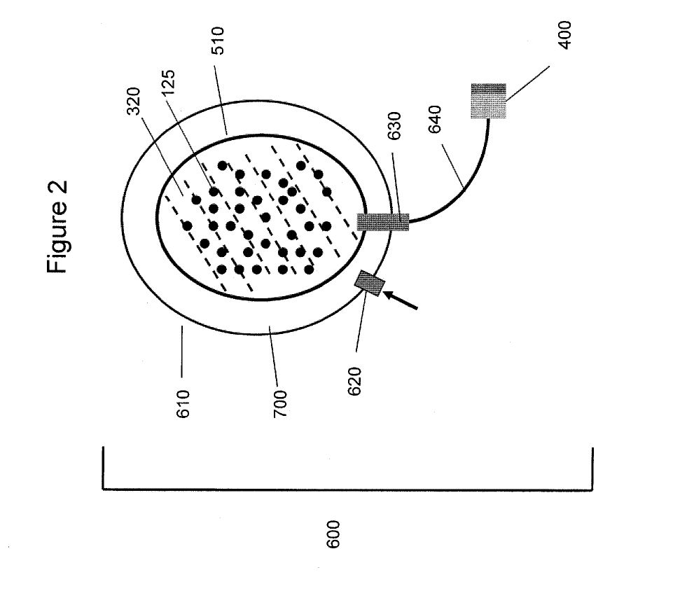

In other embodiments, delivery system 600 depicted in FIG. 2 is used to administer a suitable compressible microbubble-containing suspension to a subject. Referring to FIG. 2, this delivery system includes inner bag 510 filled with compressible suspension 320 that contains gas-filled microbubbles 125 as described above, and outer bag 610 surrounding inner bag 510. Inner bag 510 further includes port 630 for connecting to delivery device 400, as described above, via tube 640. Outer bag 610 includes port 620 through which a solution can be filled into space 700 between inner bag 510 and outer bag (or bottle) 610. Once a solution is filled into space 700, the pressure caused thereby forces compressible suspension 320 to flow into delivery device 400 through port 630 and tube 640. The delivery device (400) may consist of several standard infusion pumps found in a hospital setting, e.g., peristaltic infusion pumps or syringe pumps. During administration, inner bag 510 is placed preferably in a position (e.g., vertical), at which any trapped gas escaped from suspension 320 is accumulated at a place such at the trapped gas would not exit from inner bag 510 through port 630, thereby avoiding delivery of trapped gas to the subject.

In yet other embodiments, a microbubble-containing compressible suspension is delivered via a system comprising at least one drug delivery device for housing the suspension. The at least one drug delivery device includes two ports, one for connecting to a tube through which the suspension is delivered to a subject, and the other for releasing trapped gas, thereby avoiding its entering into the subject. The drug delivery device further includes a pressure unit as described above for applying pressure to the compressible suspension so as to control its flow rate into the subject, e.g., at least 10 to 300 mL/minute.

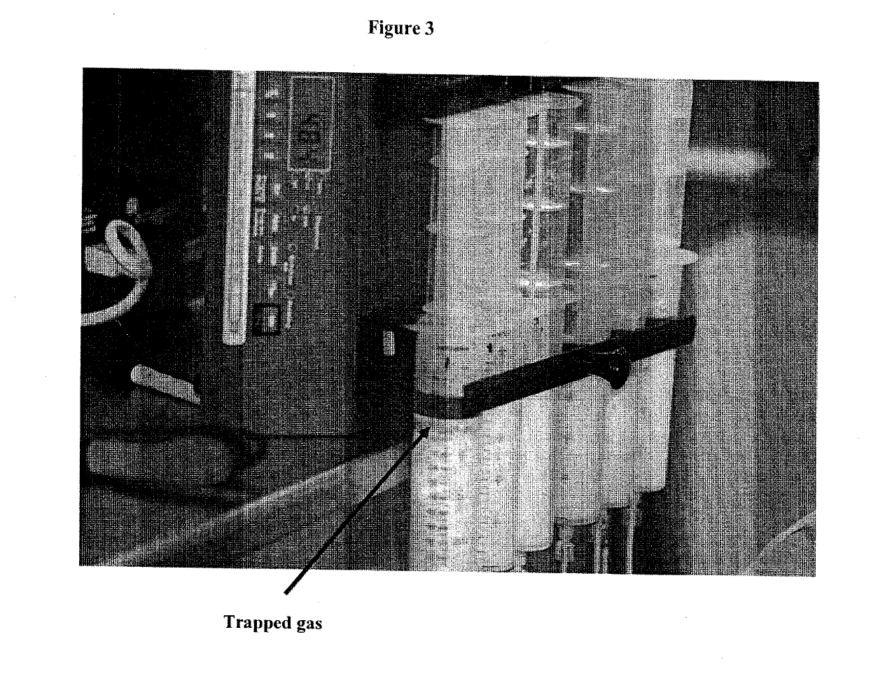

One example is shown in FIG. 3. This delivery system contains six syringes connected to a syringe pump. Each of the syringes may have a minimal volume of 100 mL. All of the syringes are placed in a vertical position to allow trapped gas to accumulate at the top of each syringe, thereby avoid delivering such trapped gas into a patient. The six syringes are collected to a tube through which the microbubble suspension is delivered into a patient at a predetermined infusion rate, which can be controlled by the syringe pump. Highly concentrated suspensions containing oxygen-filled microbubbles can be placed into each of the syringes for delivery. Such concentrated suspensions preferably have low viscosity such that they do not hold trapped gas. When containing 100 mL of a suspension having 70% oxygen by volume, the total weight of the syringe (together with the suspension) can be around 28 g.

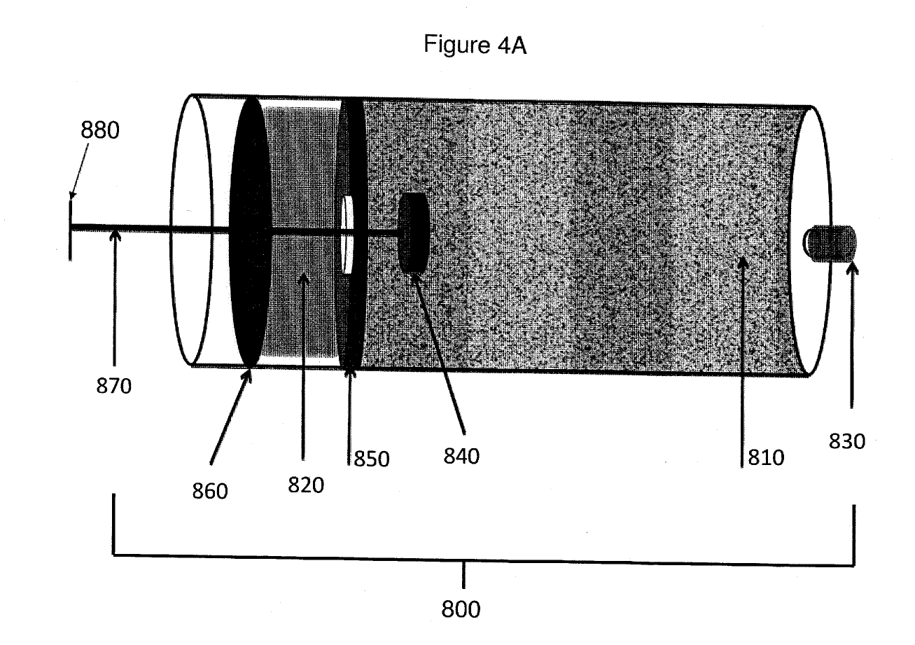

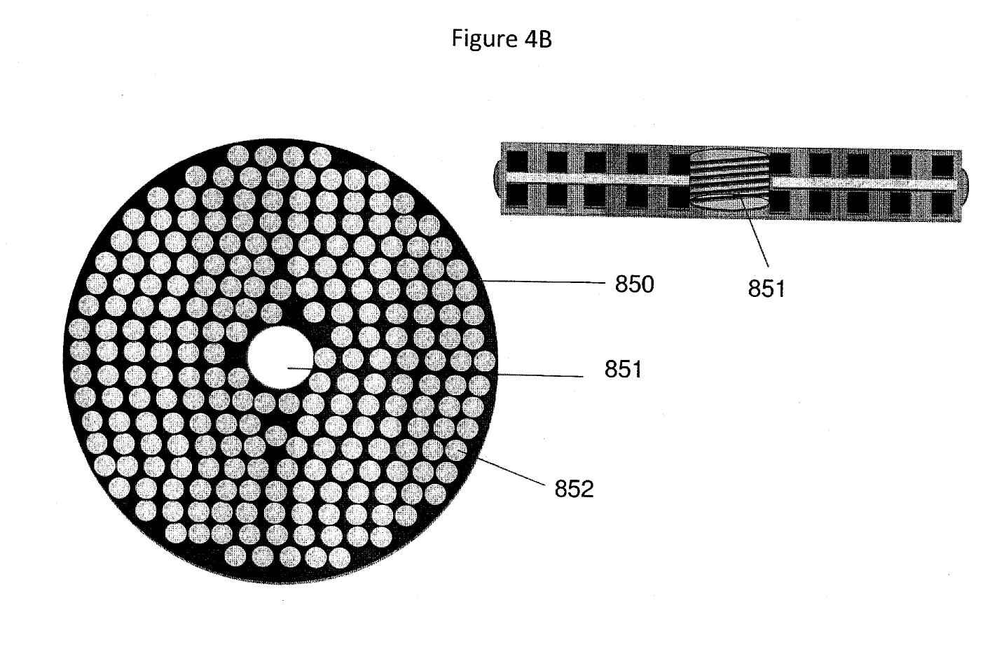

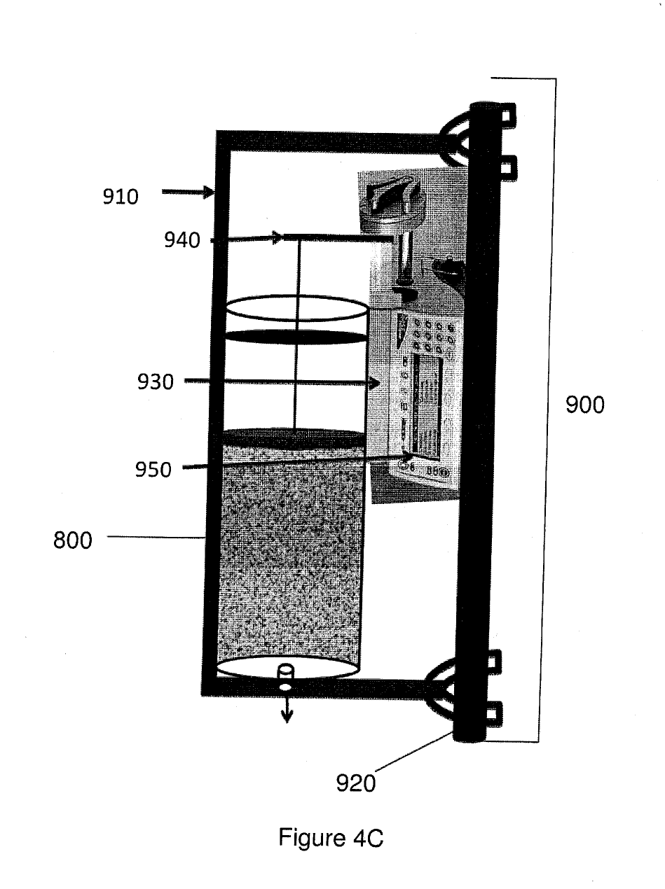

In some embodiments, syringe-based gas infusion apparatus 800, as depicted in FIG. 4A, is used to deliver gas into a patient. The entire apparatus 800 infusion can carry up to 5 liters of components in the gas, aqueous or solid phase. The outer walls of the apparatus can be made of plastic (of any composition) or glass. The outer walls can be marked with gradations providing estimates of the volume contained within the apparatus at each marking. The outlet of the apparatus will be fitted with a luer locking system that can interface with standard intravenous or intraarterial lines used in the medical setting (including the prehospital setting). It will come packaged with a gas-tight cap on the end of the syringe.

Referring to FIG. 4A, infusion apparatus 800 includes filter plate 850 to separate the apparatus into two chambers, chamber 810 and chamber 820. Chamber 810 is for housing gas or gas-filled microbubbles, either in dry form or in suspension form. For example, this chamber can contain highly viscous and concentrated microbubbles in the aqueous phase (e.g. microbubbles containing >85 mL oxygen per dL of suspension). Chamber 820 is for housing an aqueous diluent, such as normal saline, plasmalyte or lactated ringers, which can be enclosed within a suitable fragile bag such that it break easily with the motions described below but not in storage or transit. This diluent is to be used for dilute the gas or gas-filled microbubbles in chamber 810.

Filter plate 850, separating chamber 810 and 820, can be made of a firm material (such as metal or plastic). This plate contains a central hole surrounded by many small holes (i.e. a perforated disc) in each of which a filter resides. See FIG. 4B. Both the central hole and the perforations allow a liquid and a gas to pass through the plate. Filter plate 850 can be made of two identical and aligned discs containing a solid piece of filter paper wedged between them. The central hole can be fitted with a thread so that plunger disc 840 described below can screw into it.

Infusion apparatus 800 also includes plunger shaft 870 attached to plunger disc 840 and compressing disc 860. The plunger disc can be made of the same material as the filtering plate. The plunger disc can have threads to attach to the filtering plate using a twisting motion. Alternatively, the filtering plate and the plunger disc can be fitted with an apparatus which allows the two to connect by a click, or even by coming into close contact by magnetic forces. Plunger shaft 870 can have a handle at one end allowing for ease of use, specifically movement in and out of any of the chambers mentioned above, as well as twisting of the handle. The handle, which can be made of metal or plastic, optionally have a simple elbow (L-shaped) or T shaped as shown in FIG. 4A. It may also be an ergonomic circle. The shaft can pass freely through the central hole of the filtering plate.

Compressing disc 860 can be made of a solid material (such as metal or plastic or rubber). It may mirror the movements of plunger shaft 870 and plunger disc 840. It may be attached to the plunger shaft by material continuity (e.g. welding or a plastic mold) or may be attached using a threaded handle, which could be fitted with ball bearings allowing the plunger shaft to be twisted easily (for screwing of the plunger disc into the central hole of the filtering disc). When chamber 820 contains the bag mentioned above for storing the aqueous diluent, the bag can be attached broadly (all the way to the edges) to the facing aspect of the compressing disc.

The above-described infusion apparatus can be used to rapidly mix and infuse any suspension. It could be used to mix and deliver gas-filled microbubbles, including oxygen gas-filled microbubbles. The device will be easy to use in an emergency, allow for rapid administration of high volumes of fluid, and will filter out entrapped gas.

Apparatus 800 can be stored in any position and at any clinically-relevant temperature as determined by the materials contained within it. For example, the apparatus can be kept on an ambulance, in an emergency department or in an ICU. When ready for use, the apparatus can be removed from packaging. The handle of plunger shaft 870 can be depressed towards the center of the apparatus. This will break the bag containing the diluent. The handle can be further depressed towards the center of the apparatus until compressing disc 860 meets filtering plate 850. This could force the diluent to flow from chamber 820 to chamber 810 through the central hole (or less likely, through the high-resistance filtered pores), and mix with the content of chamber 810. The apparatus can be shaken vigorously for a time period determined by the contents of the chambers such that the contents can mix well to form a suspension ready for administration. The handle can then be withdrawn until the plunger disc meets the filtering plate. This may also pull back the bag in which the contents of chamber 820 were stored to above the level of the filtering disc. The plunger shaft can then be attached to the filtering plate by screwing (or snapping, etc) the plunger disc to the central hole of the filtering plate, using, e.g., a twisting motion. See FIG. 4B. Chamber 810 and chamber 820 will then be separated only by the filtered discs of the filtering plunger.

With the apparatus held vertically (the luer towards gravity and the handle away), the plunger shaft can then be depressed until the filter plate meets the gas-suspension interface. Gas accumulated above the suspension in chamber 810 (i.e., trapped gas) can therefore pass easily though the filter pores and into chamber 820, thereby avoiding delivering trapped gas into a patient. Any foam or large gas bubbles would break upon contact with the filtering plate and the gas would pass through the filtering plate. Once the aqueous phase comes into contact with the suspension, the microbubbles will become trapped within the filters residing in the perforations such that the filtering plate will become functionally occluded.

Apparatus 800 includes port 830 at the bottom of chamber 810. Before infusion, the port is covered by a cap. When a suspension formed in chamber 810 is ready for administration, the cap can be removed and the luer connected to any standard line (central or peripheral) attached to the patient. Alternatively, it could be attached to an enteric feeding tube, a pleural, peritoneal or subdural/intrathecal catheter or needle for enteric, pleural, peritoneal, cerebral or topical uses, respectively.

A tubing connecting port 830 may contain a third chamber, which can serve as a macrobubble trap. A tall column filled with a liquid or an empty, vertical tube could be used.

The apparatus can be agitated manually. More specifically, the plunger shaft can be depressed manually to inject the suspension in chamber 820 into the compartment (e.g., a vein) attached to the tip of the apparatus. Alternatively, the plunger shaft can be attached to a second apparatus designed to depress the plunger at a specific rate (see, e.g., FIG. 4C).

In the case of oxygen gas, a patient may require as much as 200 mL/minute of oxygen gas. Partial supplementation may require 50 or 100 mL/minute. Standard syringe pumps, however, administer a maximum of 300 mL/hour, or 5 mL/minute.

Gas infusion System 900, containing apparatus 800 described above, can be used to deliver gas-filled microbubbles into a patient at high volumes (higher than any currently available clinically-used medication as discussed above). As shown in FIG. 4C, apparatus 800 is mounted onto IV pole 920 via support structure 910 by, e.g., clamping, strapping or screwing onto the pole, in a manner that apparatus 800 can be adjusted vertically, horizontally, or both, e.g., using poles which are collapsible. A counterweight may be added to the opposing side to avoid tipping of standard IV poles. In this system, apparatus 800 can also be connected, via, e.g., a clamp, a syringe pump, which is available in most hospitals, for infusion control (e.g., infusion volumes and/or infusion rates).

Syringe adapter 930 can be affixed to apparatus 800 by strapping, latching, screwing or another suitable mechanism. Alternatively, the apparatus could come manufactured including a syringe adapter. The purpose of the syringe adapter is to fit into the mechanism of standard syringe pumps and permit an interface between the two. It can also serve to physically attach apparatus 800 to the syringe pump because the apparatus, which may be used to hold a number of liters, may not fit into standard syringe pumps. Plunger adapter 940 can be affixed to the plunger shaft of apparatus 800 for fitting into the plunger depressor of a standard syringe pump.

In addition, a computer device can be either installed into or connected to the syringe pump. A software modification to each infusion pump may allow healthcare workers to enter the infusion volume (based on the size of the super-syringe) and/or an infusion rate in mL/minute. Alternatively, the infusion rate could be titrated by a computer which received inputs from the patient's monitor which included oxygen saturations and increased or decreased the infusion rate to achieve a goal oxygen saturation. Cooperation of syringe pump manufactures (e.g. Baxter) may be used for this modification

### (III) Uses of Gas-Filled Microbubbles

The gas-filled microbubbles described herein can be used to deliver a gas (e.g., oxygen) into a subject, thereby treating various diseases and conditions. The term “treating” as used herein refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disease/disorder, the symptoms of the disease/disorder, or the predisposition toward the disease/disorder.

### (i) Therapeutic Applications of Oxygen-Filled Microbubbles

Suspensions containing oxygen-filled microbubbles as described herein can be used to restore the oxygen level in a patient experiencing or being suspected of experiencing local or systemic hypoxia via any of the methods described above. Thus, they have broad therapeutic utilities, including treatment of traumatic brain injury, cardiac arrest (via either intraarterial infusion or intravenous infusions), promotion of wound healing, and preservation of organs during transplant. Below are some examples.

### Cerebral Protectant During Childbirth

An effective amount of suspension containing oxygen-filled microbubbles and optionally other therapeutic agents can be administered into the subdural (or nearby) spaces during intrapartum distress so as to maintain sufficient oxygen supply to the neonate, thereby reducing the risk of cerebral damage during childbirth.

### Provide Oxygen Supplementation Via the Enteral Route

A suspension containing oxygen-filled microbubbles and, optionally, lipid nutrients or nutrients found in blood (e.g., glucose and other blood components), can be delivered via a enteral route, e.g., to a site in the abdominal cavity, such as the intestine or the peritoneum, to provide an alternate source of intestinal oxygenation and prevents or mitigates intestinal ischemia, which may contribute to necrotizing enterocolitis, a leading cause of pediatric morbidity and mortality in preterm infants. This can also benefit prematurely born infants as it may decrease toxicity to premature lungs, prevents retinopathy of prematurity, and also provides lipid nutrition at the same time. In addition, it may be used in adults such as COPD patients, who require supplemental oxygen for some reason. It may also provide an alternative method of providing supplemental oxygen to critically ill patients such as ARDS patients, in whom increasing oxygen delivery through the lungs may be prohibitively injurious.

### Preservation of Organ and Blood In Vitro

Low blood oxygen tensions may contribute to the blood storage defect, causing cells within the plasma to generate lactate and toxins, which may decrease the therapeutic value of transfused blood and diminish its shelf life. Oxygen-filled microbubbles may be added to a blood sample periodically to prolong in vitro blood storage. In an explanted organ, a suspension containing oxygen-filled microbubbles can be delivered into a blood vessel in an organ to provide oxygen supply, thereby ameliorating tissue damage due to hypoxia. This is particularly useful in preserving organs to be used in transplantation.

In addition, oxygen-filled microbubbles can be added to a blood sample periodically to prolong in vitro blood storage.

### Promote Wound Healing

Delivery of a suspension containing the oxygen-filled microbubbles described herein to a wound site or a site nearby a wound can provide a continuous supply of oxygen to the wounded tissue, which is essential to the healing process. Thus, this approach benefits healing of a wound, such as that associated with a disease or disorder (e.g., diabetes, peripheral vascular disease, or atherosclerosis). In some embodiments, the suspension is prepared as a topical formulation for treating external wounds.

### Improve Efficacy of Tumor Radio Therapy and Reduce Side Effects Caused Thereby

Tumor radio therapy often damages non-cancerous tissues nearby a tumor site. Applying an effective amount of a suspension containing oxygen-filled microbubbles described herein, when infused either locally or systemically, can reduce such damage by increasing the oxygen content of a local tumor environment. In addition, it also can increase the effects of ionizing radiation delivered to the tumor, thereby improving efficacy of a radio therapy. In some embodiments, the suspension is delivered directly to a tumor site. In others, the suspension can be administered to a site nearby a tumor.

### Ameliorate Sickle Cell Crisis

Sickle cell crisis refers to several independent acute conditions occurring in patients with sickle cell anemia, including acute chest syndrome (a potentially lethal condition in which red blood cells sickle within the lungs and lead to necrosis, infection and hypoxemia), vaso-occlusive crisis (i.e., obstruction in circulation caused by sickled red blood cells, leading to ischemic injuries), aplastic crisis (acute worsening of the baseline anemia in a patient, causing pallor, tachycardia, and fatigue), splenic sequestration crisis (acute, painful enlargements of the spleen), and hyper haemolytic crisis (acute accelerated drops in haemoglobin level). Administering an effective amount of suspension containing oxygen-filled microbubbles as described herein to a sickle cell anemia patient or a subject suspected of having the disease can reduce sickle cell crisis, in particular, vaso-occlusive crisis.

### Improve Anti-Infective Activity of Immune Cells

Containing lipid, oxygen-filled microbubbles can be preferentially taken up by lymphocytes of varying types, including macrophages so as to raise intracellular oxygen tension. This may potentiate lymphocyte killing of microbial agents by enabling superoxide dismutase and the production of intracellular free radicals for microbicidal activity without causing resistance.

### Minimize Organ Injury During Cardiopulmonary Bypass in Adults, Children, and Neonates.

During cardiopulmonary bypass operations, the heart must be cross-clamped (i.e. no oxygen delivery) and cooling/protective agents reduce myocardial oxygen consumption. Use of oxygen-filled microbubbles to add a small amount of oxygen supply on a continuous basis to organs or to the blood used to deliver the cold cardioplegia solution would better protect the heart and prevent post-cardiac bypass injury. The majority of the oxygen-filled microbubbles is gas, which could be consumed by the myocardium, leaving only a lipid shell and a small amount of carrier, if any. This is important because a large volume of perfusate cannot be used due to obscuration of the surgical field. This may provide a way to keep a clean surgical field while still providing oxygen to the myocardium, with or without hemoglobin as an intermediary.

### Oxygenate Venous Blood in Myocardial Infarction Patients

During a heart attack (myocardial infarction), an arterial thrombus prevents perfusion and therefore oxygen delivery to a selected region of myocardium. Perfusing the right atrium (through an intravenous injection) with highly oxygenated blood, via delivery of oxygen-filled microbubbles, and providing a high coronary sinus pressure via a high right atrial pressure can back-perfuse a region of ischemic myocardium via the coronary sinus and venous plexus of the heart. The majority of the volume of the injectate (i.e., gas) will be consumed and disappear, allowing a continuous infusion into a dead-end space (i.e. a venous plexus feeding a region of myocardium previously fed by a thrombosed coronary artery, whether partially or completely obstructed. The thin-walled atrium may directly absorb oxygen from the oxygen-rich right atrial blood. In practice, using oxygen-filled microbubbles can be an easy way to perfuse the heart with oxygen rich blood during acute coronary syndrome. For example, the oxygen-filled microbubbles can be delivered using an occlusive balloon catheter blown up in the coronary sinus with a power-injection of oxygen-rich suspension into the coronary sinus such that the suspension could flow retrograde throughout the heart, including the region affected by the coronary thrombus (because there would be no clot on the venous side).

### Reduce Cardiac Arrhythmia During Coronary Angiography

Cardiac arrhythmia, even fatal arrhythmia, is a common adverse effect during coronary angiography in both adults and children for diagnostic or therapeutic purposes. Using an oxygen-filled microbubble suspension 20 mL/dL oxygen, mixed with a contrast agent, allows for sustained oxygen delivery to sick myocardium during a selected injection of a coronary artery and prevents a substantial number of adverse events and deaths from these risky procedures.

### Replace Blood During Bloody Procedures or in Early Resuscitation in Trauma

A suspension containing oxygen-filled microbubbles capable of translocating oxygen directly to mitochondria can be used as “blood replacement” during bloody procedures or in the early resuscitation in trauma. This would of course be a temporizing procedure such that the ‘blood’ lost via a bleeding source (e.g. the back during a spinal fusion, other arteries during many bloody procedures) would contain mostly non-blood components. The majority or all of the blood could be removed at the beginning of an operation and the body can be perfused with oxygen-filled microbubble suspension (which also contains a buffer for the absorption of carbon dioxide, energy substrates such as glucose, and clotting factors such as platelets, FFP and cryoprecipitate) during the operation. Once the bloody portion of the procedure was near the end, the blood could be replaced, and the perfusate of oxygen-filled microbubbles could quickly go away due to absorption of oxygen gas and renal filtration (or mechanical ultrafiltration) of the diluent. When necessary, suspensions containing ˜90-95 mL of oxygen gas per dL of suspension are used given the prolonged time (hours) of providing for the body's entire oxygen consumption.

### Treat Pulmonary Hypertension

Perfusion of the venous system, and therefore the pulmonary arteries and arterioles, with ‘blood’ rich in oxygen, nitric oxide, or other gaseous vasodilators can more effectively relax the pulmonary arterioles (putatively a major contributor to the pathology of pulmonary hypertension). This would be most effective during a pulmonary hypertensive crisis, a potentially fatal event in which high pulmonary pressures cause a decrease in blood flow to the left heart and decreased cardiac output. Accordingly, a venous injection of a suspension containing oxygen-filled microbubbles can quickly reverse the process. This approach could be more effective than delivering oxygen to the lungs via inhalation because of its exposure to the pulmonary arterioles, which are the farthest point in the circulation from the pulmonary capillaries.

### Treat Pulmonary Embolus

In near-fatal pulmonary embolus a defect could be created in the atrial septum to permit the flow of venous blood across the atrial septum to allow filling of the left heart (a Rashkind balloon atrial septostomy) from the right heart, bypassing the lungs temporarily. In this setting, a suspension containing oxygen-filled microbubbles can be used to oxygenate blood, thereby permitting time and clinical stability for a surgical thrombectomy, catheter based interventions or medical therapies to be applied to the clot.

### Treat Carbon Monoxide Poisoning

Patients (including soldiers) with severe carbon monoxide poisoning are currently treated with hyperbaric oxygen. This is an expensive and scarce resource, and is impractical for unstable patients due to the technical constraints of the hyperbaric chamber itself. The oxygen-filled microbubbles described herein can be used to create hyperbaric oxygen conditions (i.e. the oxygen content of the blood under hyperbaric conditions is 22-24 mL/dL versus 20 at atmospheric pressure). More specifically, use of an oxygen-filled microbubble suspension containing 60-80 mL oxygen/dL of suspension can displace carbon monoxide from hemoglobin and restore normal hemoglobin function as occurs in the hyperbaric chamber. This would obviate the need for a hyperbaric chamber, allow for the cotemporaneous treatment of multiple patients with carbon monoxide poisoning (e.g. terrorist attacks, house fires, soldiers), the treatment of ICU patients with CO poisoning, and permit the rapid reversal of CO poisoning at or near the point of injury (e.g. at the scene of a fire).

### Reduce Injury Caused by Low Systemic Blood Oxygen Saturation

There are many congenital heart lesions in which desaturated blood (from the body) and oxygenated blood (from the lungs) mix in the heart. In some instances, e.g., immediately after a Norwood operation or unrepaired D-transposition of the great arteries, the degree of mixing or the degree of pulmonary blood flow causes the systemic saturations to be extremely low such that the body develops acidosis and organ injury. In these patients, raising the oxygen tension of the systemic venous return by even a small amount would raise the systemic oxygen saturations significantly (due to mixing). This would avert a large number of patients who currently are placed on ECMO for even a few days for this reason.

### Resuscitation in Obstructed Systemic-Pulmonary Shunts

Several congenital heart lesions (e.g. hypoplastic left heart syndrome) are initially treated with a small tube graft from the innominate artery or the right ventricle to the pulmonary artery. The acute obstruction of these shunts (usually a B-T shunt) causes death within minutes and is an important cause of interstage mortality for these children. The availability to oxygenate the venous blood in these patients, using the oxygen-filled microbubbles described herein, would allow even a paramedic to effectively resuscitate a patient in need with oxygenated blood. This could also prevent death in a substantial number of hospitalized patients in hospitals with or without the ability to rapidly place a patient onto ECMO

### Transport of Neonates

Similarly, newborns with congenital heart disease can have diseases that cause profound cyanosis and organ injury. For example, patients with D-transposition of the great arteries receive systemic arterial blood flow from the right ventricle, blood flow which is not exposed to the lungs at all. In patients with inadequate mixing at the atrial level, profound cyanosis can cause organ injury and death. These patients could be stabilized and transported to definitive care by oxygenating the venous return via infusion of oxygen-filled microbubbles. Patients with obstructed pulmonary venous return, representing the only true pediatric congenital heart emergency, could be stabilized by creation of an atrial septal defect and oxygenation of venous return as discussed above.

### Provide Inotropic Support

Myocardium extracts a higher proportion of oxygen from the blood than any other organs. In post-cardiac bypass or post-myocardial infarction patients (exhibiting tissue edema and mitochondrial dysfunction), a catheter placed into the coronary root may allow delivery of oxygen-filled microbubbles, thereby supersaturating the coronary blood flow and provide a novel route of inotropic support different from all current inotropic methods, all of which rely on the beta receptor. This approach could provide an effective inotropic supplement, especially to those patients with downregulated beta receptors.

### Treat Multi-Organ Dysfunction Syndrome

Use of an oxygen-filled microbubble suspension with high oxygen concentration can be used to achieve extremely high oxygen tensions at the capillary level with or without hemoglobin. This would enhance the uptake of oxygen by dysfunctional mitochondria or through an inflamed endothelium.

### (Ii) Therapeutic Applications for Microbubbles Encapsulating Non-Oxygen Gas

Delivery of a pharmaceutically acceptable gas other than oxygen can confer various therapeutic benefits. For example, isoflorane-filled microbubbles can be delivered to a patient having or suspected of having asthma for treating the disease. In another example, microbubbles filled with an insoluble gas (e.g., nitrogen or a noble gas) can be used as a volume expander. Particularly, microbubbles having a size of 1-5 microns do not pass through gap junctions and thereby serve as an excellent volume expander. Moreover, gaseous sedatives can be delivered via gas-filled microbubbles to achieve a quick effect.

In addition to therapeutic applications, gas-filled microbubbles can also be used for non-therapeutic purposes, e.g., as MRI contrast agents, fuel additives, or research tools for defining the volume of oxygen exposed to an environment.

Other utilities of gas-filled microbubbles, particularly oxygen-filled microbubbles, are described in US 2009/0191244, the entire content of which is incorporated herein by reference.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1

### Preparation of Oxygen-Filled Microbubble Suspensions

A suspension containing O₂-filled microbubbles was manufactured using the apparatus described in Swanson et al., 2010 with modifications. Briefly, an aqueous suspension containing one of the phospholipids and one of the stabilizing agents listed in Table 1 below was prepared by gentle mixing in normal saline.

TABLE 1
Constitutes in Gas-filled Microbubbles

| | | | | Stabilizing |
|---|---|---|---|---|
| | | Lipid | | agent |
| | | Concen- | | fraction |
| Suspension | | tration | Stabilizing | (mol % of |
| Number | Lipid | (mg/ml) | agent | Lipid) |
| 1 | DPPC (16:0 PC) | 7.5 | PEG40S | 10 |
| 2 | DPPC (16:0 PC) | 7.5 | PEG40S | 20 |
| 3 | DPPC (16:0 PC) | 7.5 | BRIJ S 100 | 10 |
| 4 | DPPC (16:0 PC) | 7.5 | BRIJ S 100 | 20 |
| 5 | DSPC (18:0 PC) | 7.5 | PEG40S | 10 |
| 6 | DSPC (18:0 PC) | 7.5 | PEG40S | 20 |
| 7 | DSPC (18:0 PC) | 7.5 | BRIJ S 100 | 10 |
| 8 | DSPC (18:0 PC) | 10 | DSPE- | 20 |
| | | | PEG5000 | |
| 9 | DSPC (18:0 PC) | 7.5 | DSPE- | 20 |
| | | | PEG2000 | |
| 10 | DSPC (18:0 PC) | 10 | Poloxamer 188 | 10 |
| 11 | DSPC (18:0 PC) | 7.5 | BRIJ S 100 | 20 |
| 12 | DAPC (20:0 PC) | 7.5 | PEG40S | 10 |
| 13 | DAPC (20:0 PC) | 7.5 | BRIJ S 100 | 10 |

The suspensions were infused through three parallel sonicators fitted with continuous flow attachments, the inside of which maintained a pure oxygen environment. Care was taken to ensure that energy was focused on a small region of lipid suspension, creating size-limited particles. The resultant suspension then flowed into a rise column for defoaming, followed by concentration via serial centrifugation at 1500 rpm for 10 minutes.

Five liters of a suspension containing DSPC (10 mg/mL) and 10 mol % Poloxamer 188 or Brij® S 100 were mixed with normal saline and pumped at a constant rate through three sonicators fitted with continuous flow attachments, along with oxygen gas set at 60 mL/minute into each attachment. This produced a solid white output. The suspension flowed passively into one of three 2 liter, custom built, water jacket cooled and oxygenated column for size isolation by floatation and foam exclusion. The column was filled to capacity, then intentionally overflowed, floating the foam out of the vent and into a collection chamber. The column was filled until 300-400 mL of foam were collected in the collection chamber. The column was then allowed to stand until two demarcating lines are noted. One denoted a foam top was formed within 1-2 minutes at the top of the column and the second demarcating line formed more slowly (over 5-8 minutes) and denoted rapidly buoyant (>8-10 microns, with bubbles measuring as large as 50-100 microns) from less buoyant particles (<8-10 microns). The suspension below this line was collected and centrifuged at 4 degrees, 500 RPM, for 15 minutes. The resultant microparticle cakes were combined, placed into a 140 mL syringe, and stored at 4 degrees. The just-described manufacturing process took place over a 2 day period.

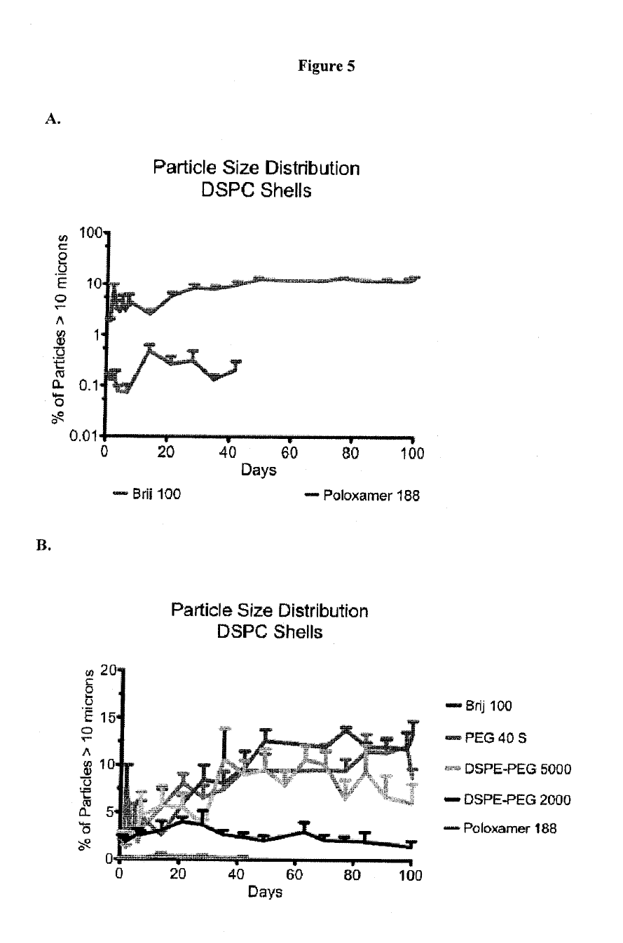

For oxygen-filled microbubbles containing DSPC and 10 mol % Brij® S 100 or DSPC and 10 mol % Poloxamer 188, their size distribution determined by optical scatter exhibited a mean diameter of 2.30±1.57 microns. Light microscopy of the suspensions containing DSPC/BRIJ or DSPC/Poloxamer 188 microbubbles exhibited a polydisperse size distribution. Transmission electron microscopy of the microbubbles demonstrated that each of these microbubbles has a gas core surrounded by a lipid bilayer and a PEGylated brush border. The mean oxygen content of the suspensions by mass differential was 71.3±10 mL per dL. See also FIG. 5.

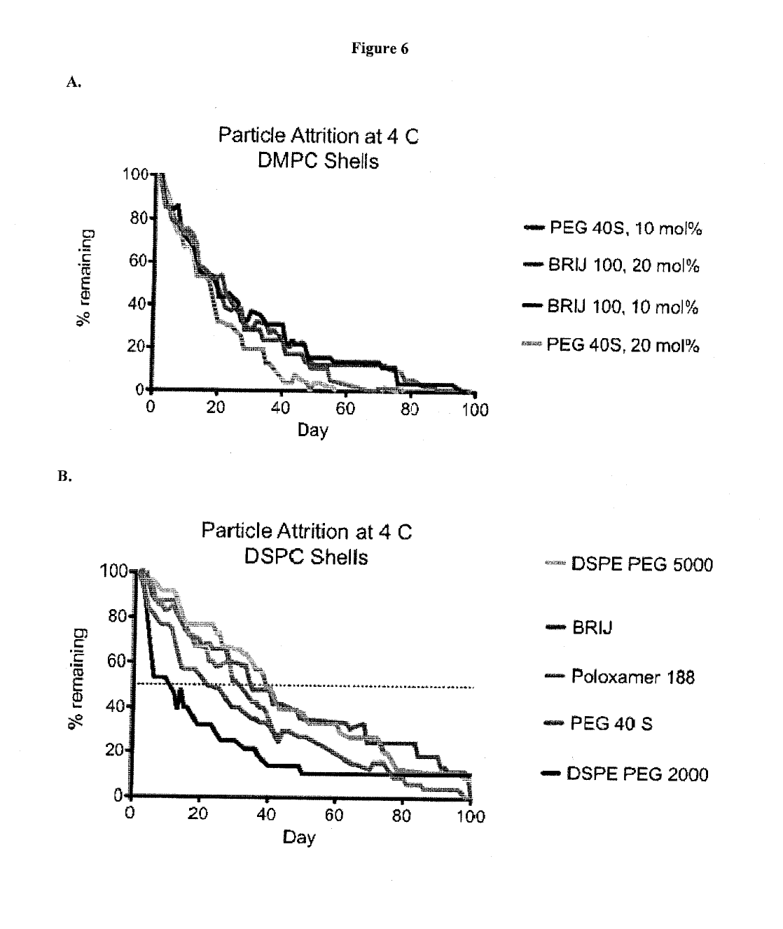

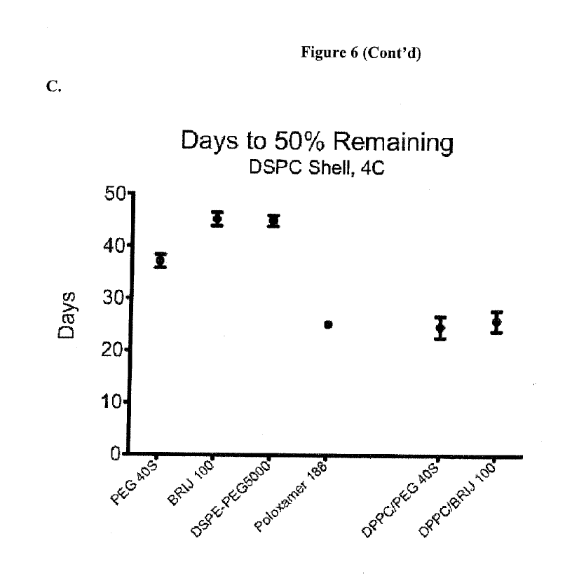

Stabilities of the microbubble suspensions described above were examined following the method described in US 2009/0191244. Briefly, a rise column was used as a defoaming chamber. The yield was increased by placing a drainage tube from the bottom part of the rise column back into the precursor container such that the dependent-most portion of the column drained and was recycled back through the sonicator. In this way, the yield of microbubbles was optimized. After allowing the microbubbles to recycle continuously for 10 minutes, the drainage cannula was clamped and the rise column was filled to the top with microbubbles, expelling the foam from the top of the column. The results obtained from this study are shown in FIG. 6.

### Example 2

### Restore Oxygen Supply in Asphyxial Rabbits with Oxygen-Filled Microbubble Suspensions

Adult New Zealand white rabbits were premedicated with Midazolam 0.1 mg/kg IV followed by Ketamine 10 mg/kg IV. These rabbit were also administered with Fentanyl 100 micrograms and Pancuronium 0.1 mg/kg IV. Fentanyl 100 micrograms and Pancuronium 0.5 mg were repeated as needed for movement or perceived discomfort, and recorded on the attached flowsheet. A baseline infusion of Fentanyl was administered at 30 microgram/kg/hour of Fentanyl, titrated to animal comfort based on pupillary examination and hemodynamics.

Following sedation, the rabbits were endotracheally intubated, instrumented, paralyzed, and confirmed by auscultation and end tidal CO₂. The animal was then placed on a Servo I ventilator, ventilated according to the settings recorded on the flowsheet, and titrated to keep tidal volumes 10 mL/kg and end tidal CO₂ in the low 20s.

A continuous oxygen tension probe (Oxford Optronix) was placed into the femoral artery along with arterial and venous lines of each rabbit. Cutdowns were accomplished for placement of the catheters using the well-known Seldinger technique. There were approximately 5 mL EBL from line placement, 4 French Cordis sheath in the left femoral vein for injections, 22 gauge arterial line in the left femoral artery for blood gas monitoring, and 22 gauge arterial line in the right femoral artery for continuous PaO₂ monitoring and ABP monitoring.

Following placement of the arterial line, the blood pressures were noted to be in the 80/40s, though there was reasonable acid base balance, the baseline heart rate was 300s, resolving to the 260s with sedation. Baseline labs included complete blood count, thromboelastography, coagulation profile, H-index, and lactate dehydrogenase. Three baseline blood gases were recorded. The VO₂ was 20 mL/minute at the beginning of the experimentation.

Following baseline measurements, the endotracheal tube was clamped with infusion of an oxygen-filled microbubble suspension as described in Example 1 above at a rate equivalent to the animal's minute oxygen consumption as listed on the flowsheet. Control animals received oxygenated crystalloid at comparable rates. A clamp was placed on the endotracheal tube when the suspension filled the tubing of the Cordis. The infusion was titrated to maintain PaO₂ (based on the PO₂ probe and confirmed by serial arterial blood gases) between 26 and 35 mm Hg. The infusion was titrated down slowly over the time period, but the tested animals never became hyperoxic. The animals were clamped for 15 minutes with no loss of pulsatility and extremely stable hemodynamics. The animals were unclamped at 15 minutes and 15 seconds as an empiric endpoint.

Measured endpoints included time to loss of aortic pulsations, arterial blood gas, and co-oximetry each minute during asphyxia, continuously recorded vital signs and arterial oxygen tension, markers of organ injury, hemolysis and coagulation parameters were drawn prior to and 1 hour following experimentation. All PaO₂/FiO₂ measurements were taken on 21% oxygen.

The animals had stable hemodynamics following unclamping. A mild amount of ectopic atrial or ventricular beats was noted. The pH had reached a nadir of 7.20. The animals were ventilated on 40% oxygen except around the timing of the follow up ABG, which was taken on 21% FiO2. 45 minutes into the observation period, the animals infused with oxygen-filled DSPC/Poloxamer microbubbles were noted to move spontaneously and these movements seemed appropriate. This represents metabolism of the Pancuronium 0.1 mg/kg dose within 60 minutes of last administration.

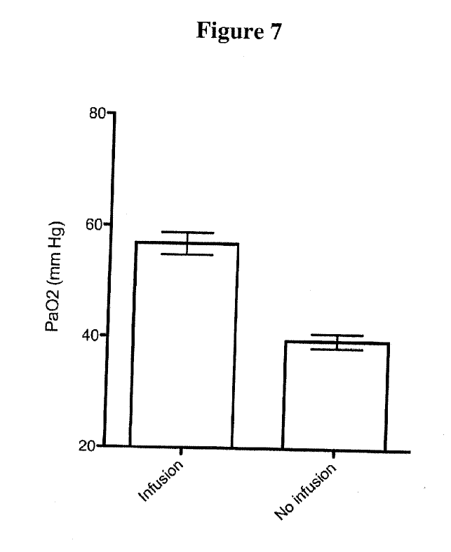

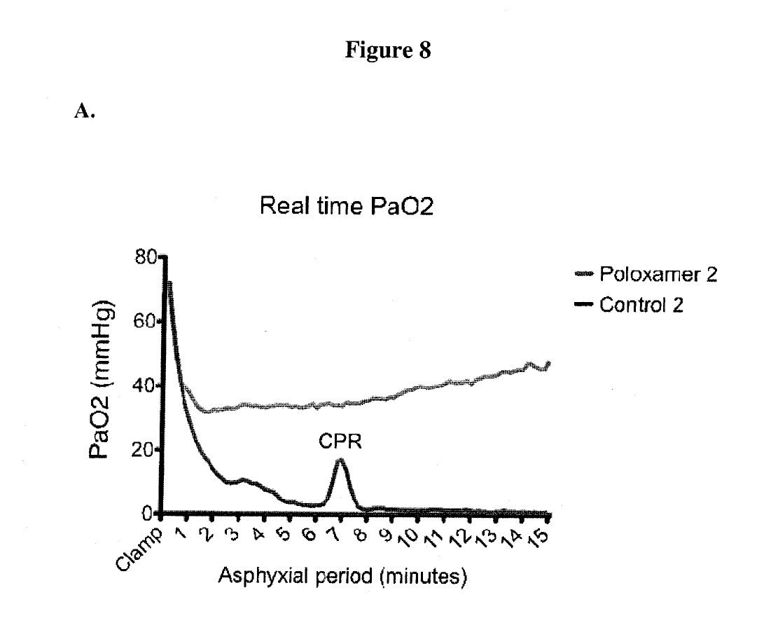

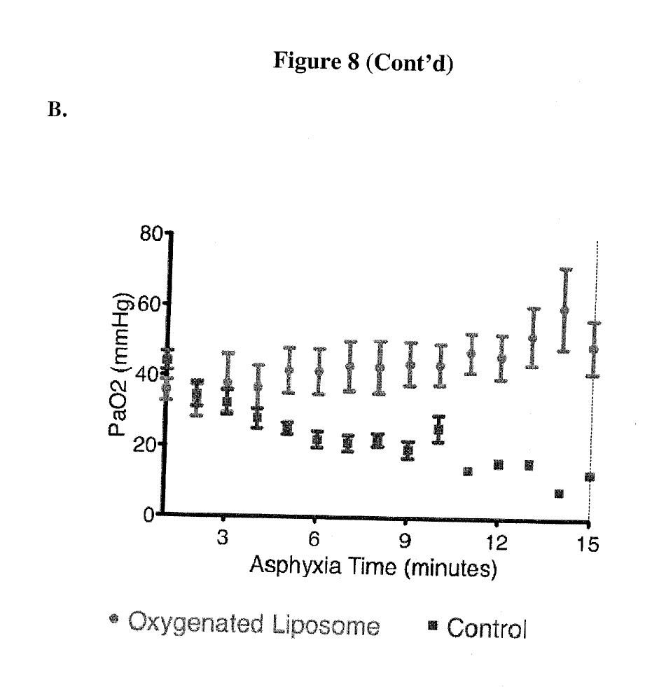

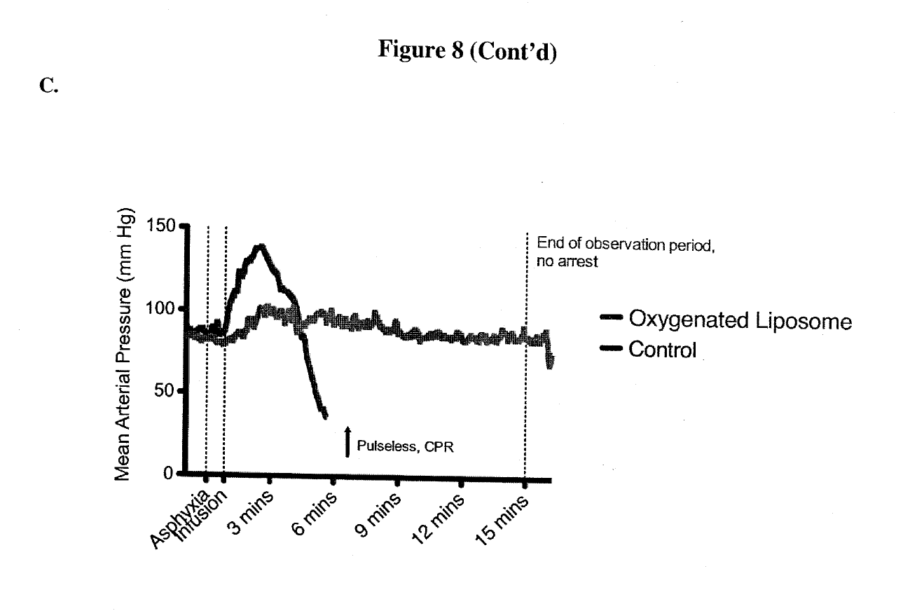

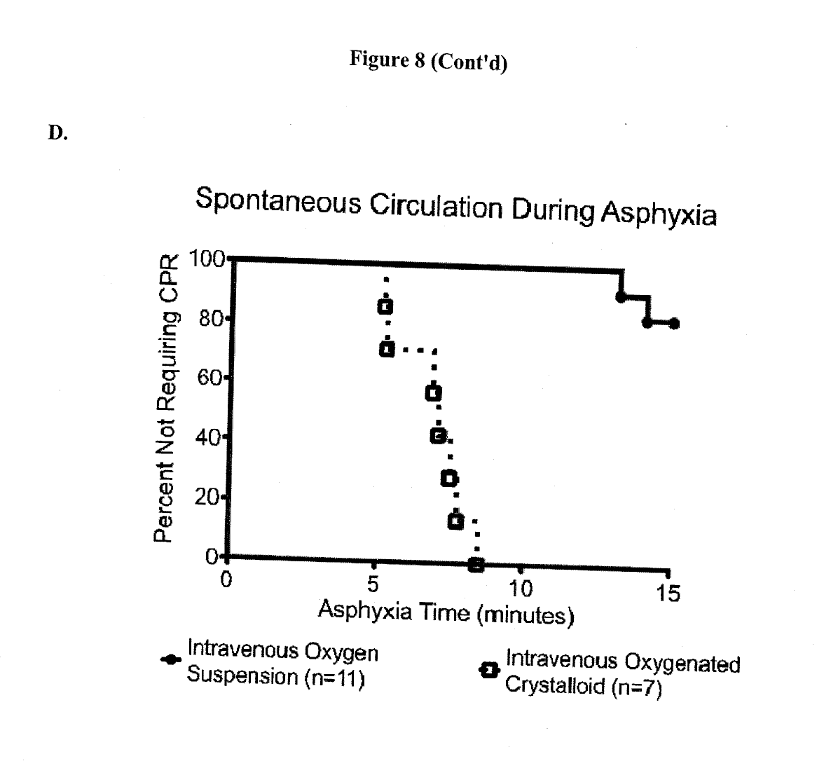

As shown in FIG. 7, rabbits treated by infusion of oxygen-filled microbubbles (hand delivered) showed a much higher oxygen saturation level than untreated rabbits. In rabbits suffering from asphyxia, those treated with a suspension containing oxygen-filled microbubbles that contains poloxamer 188 exhibited a steady and even increasing PaO₂ level throughout the asphyxial period, while untreated rabbits showed a rapid decrease in PaO₂ level even though CPR were performed on these untreated rabbits. FIG. 8, Panels A and B. Mean arterial blood pressure was preserved in the rabbits treated with intravenous oxygen throughout the period of asphyxia. Almost all rabbits treated with oxygen-filled microbubbles showed spontaneous circulation during asphyxia, while almost all rabbits treated with oxygenated crystalloid by intravenous injection required CPR within 8.5 minutes of asphyxia. FIG. 5, Panel D. The rabbits treated with oxygen-filled microbubbles exhibited a significantly lower incidence of cardiac arrest during the asphyxial period (20% versus 100%, p<0.001), while the rabbits treated with placebo universally sustained profound hypotension and cardiac arrest within 8.5 minutes of asphyxia. Measured arterial oxygen tensions were higher amongst animals treated with intravenous oxygen suspensions when compared with oxygenated crystalloid infused at the same rate.

One rabbit treated with oxygen-filled microbubbles survived for around 105 minutes following the application of the clamp (90 minute observation period) with very stable hemodynamics and rhythm. A pupillary exam showed a brisk blink and pupillary constrictive response. Followup labs were drawn at 1 hour and the animal was sacrificed using FatalPlus after the observation period.

Following FatalPlus, the rabbit was open widely for observation. Photographs and histologic specimens were taken for further inspection. The results indicate that its heart was normal in appearance and did not exhibit evidence of subendocardial ischemia on gross examination and cross-section. The lungs exhibited only minimal bruising around the ribs. There was no evidence of injury to the liver or kidney, and samples of these were taken. There was evidence of lipemic serum on inspection of the blood following centrifugation. It should be mentioned that the serum collected from this animal was kept on ice until separated and frozen at −80° C. It urine output was quantified to be 92 mL. No visible hemolysis was observed.

In sum, the results from this study indicate that infusion of the oxygen-filled microbubbles described herein were able to effectively maintain life and stabilize hemodynamics.

### Example 3

### Preparation of Oxygen-Filled Microbubble Suspensions Using Various Combinations of Lipids and Stabilizing Detergents

Suspensions containing O2-filled microbubbles were successfully prepared using the lipid and stabilizing detergent combinations shown in Table 2 below:

TABLE 2
Combinations of Lipid and Stabilizing Agents for Preparing Oxygen-Filled
Microbubbles

| | Component 1 | Component 2 | Component 3 | Component 4 | Component 5 |
|---|---|---|---|---|---|
| A | DSPC 10 mg/mL | F68 10 mg/mL | Cholesterol | | |
| | | | 5 mg/mL | | |
| B | DSPC 10 mg/mL | | | | |
| C | DSPC 20 mg/mL | Cholesterol | | | |
| | | 10 mg/mL | | | |
| D | DSPC 20 mg/mL | F108 20 mg/mL | Cholesterol | PVP 20 mg/mL | |
| | | | 10 mg/mL | | |
| E | DSPC 10 mg/mL | F68 20 mg/mL | Cholesterol | | |
| | | | 10 mg/mL | | |
| F | DSPC 20 mg/mL | F108 20 mg/mL | NaDOC | | |
| | | | 2 mg/mL | | |
| G | DSPC 20 mg/mL | F108 20 mg/mL | NaDOC | F68 20 mg/mL | |
| | | | 2 mg/mL | | |
| H | All components of G | Cholesterol | PVP 20 mg/mL | | |
| | | 10 mg/mL | | | |
| I | DSPC 10 mg/mL | F68 20 mg/mL | PVP 20 mg/mL | | |
| J | DSPC 10 mg/mL | PVP 20 mg/mL | NaDOC | | |
| | | | 2 mg/mL | | |
| K | DSPC 20 mg/mL | F68 20 mg/mL | PVP 20 mg/mL | | |
| L | DSPC 20 mg/mL | F108 20 mg/mL | PVP 20 mg/mL | Cholesterol | |
| | | | | 10 mg/mL | |
| M | DSPC 10 mg/mL | F108 20 mg/mL | NaDOC | | |
| | | | 2 mg/mL | | |
| N | DSPC 10 mg/mL | F108 20 mg/mL | NaDOC | Cholesterol | F68 20 mg/mL |
| | | | 2 mg/mL | 10 mg/mL | |
| O | All components of N | PVP 20 mg/mL | | | |
| P | DSPC 10 mg/mL | PVP 20 mg/mL | Gelatin 6 mg/mL | | |
| Q | DSPC 10 mg/mL | F108 20 mg/mL | Gelatin 6 mg/mL | | |
| R | All components of Q | F68 20 mg/mL | Cholesterol | | |
| | | | 10 mg/mL | | |
| S | DSPC 20 mg/mL | F108 20 mg/mL | Cholesterol | Gelatin | |
| | | | 10 mg/mL | 6 mg/mL | |
| T | DSPC 20 mg/mL | Cholesterol | Gelatin 6 mg/mL | NaDOC | PVP 20 mg/mL |
| | | 10 mg/mL | | 2 mg/mL | |
| U | DSPC 20 mg/mL | Gelatin 3 mg/mL | Cholesterol | | |
| | | | 10 mg/mL | | |
| V | DSPC 1 mg/mL | F68 10 mg/mL | Cholesterol | | |
| | | | 5 mg/mL | | |
| W | DSPC 5 mg/mL | F68 10 mg/mL | Cholesterol | | |
| | | | 5 mg/mL | | |
| X | DSPC 5 mg/mL | F68 10 mg/mL | Cholesterol | | |
| | | | 10 mg/mL | | |
| Y | DSPC 5 mg/mL | F68 5 mg/mL | Cholesterol | | |
| | | | 5 mg/mL | | |
| Z | DSPC 10 mg/mL | Brij 5.9 mg/mL | | | |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

1. A gas-filled microbubble, comprising a lipid membrane encapsulating a gas core, wherein the lipid membrane contains (a) 1,2-disteroyl-sn-glycero-3-phosphocholine (DSPC) or dipalmitoylphosphatidylcholine (DPPC), and (b) one or more stabilizing detergents selected from the group consisting of poloxamer 188, a poloxamer having a molecular weight lower than that of poloxamer 188, Pluronic F108, Pluronic F127, polyoxyethylene (100) stearyl ether, cholesterol, gelatin, polyvinylpyrrolidone (PVP), and sodium deoxycholate (NaDoc).

2. The gas-filled microbubble of claim 1, wherein the one or more stabilizing detergents are poloxamer 188, polyoxyethylene (100) stearyl ether, cholesterol, Pluronic F108, and PVP.

3. The gas-filled microbubble of claim 1, wherein the microbubble has a diameter of 1 to 10 microns.

4. (canceled)

5. The gas-filled microbubble of claim 1, wherein the gas core consists of oxygen, carbon dioxide, carbon monoxide, nitric oxide, inhalational anesthetic, hydrogen sulfide, or a mixture thereof.

6. (canceled)

7. The gas-filled microbubble of claim 1, wherein the lipid membrane contains:
DSPC and poloxamer 188;
DSPC and polyoxyethylene (100) stearyl ether;
DSPC and cholesterol;
DSPC, poloxamer 188, and PVP, or
DSPC, Pluronic F108, PVP, and cholesterol.

8. A microbubble suspension, comprising gas-filled microbubbles suspended in an aqueous solution, each of the gas-filled microbubbles containing a lipid membrane encapsulating a gas core, wherein the lipid membrane contains (a) 1,2-disteroyl-sn-glycero-3-phosphocholine (DSPC) or dipalmitoylphosphatidylcholine (DPPC), and (b) one or more stabilizing detergents selected from the group consisting of poloxamer 188, a poloxamer having a molecular weight lower than that of poloxamer 188, Pluronic F108, Pluronic F127, polyoxyethylene (100) stearyl ether, cholesterol, gelatin, polyvinylpyrrolidone (PVP), and sodium deoxycholate (NaDoc).

9. The suspension of claim 8, wherein the one or more stabilizing detergents are poloxamer 188, polyoxyethylene (100) stearyl ether, cholesterol, Pluronic F108, and PVP.

10. The suspension of claim 8, wherein at least 50% of the microbubbles have diameters of 1 to 10 microns.

11. (canceled)

12. The suspension of claim 8, wherein the gas core consists of oxygen, carbon dioxide, carbon monoxide, nitric oxide, inhalational anesthetic, hydrogen sulfide, or a mixture thereof.

13. (canceled)

14. The suspension of claim 8, wherein the suspension contains at least 60% oxygen by volume.

15-16. (canceled)

17. The suspension of claim 8, wherein the lipid membrane contains:
DSPC and poloxamer 188;
DSPC and polyoxyethylene (100) stearyl ether;
DSPC and cholesterol;
DSPC, poloxamer 188, and PVP, or
DSPC, Pluronic F108, PVP, and cholesterol.

18. A method for delivering a gas into a subject, the method comprising administering to a subject in need thereof an effective amount of compressible suspension containing gas-filled microbubbles, each of which contains a lipid membrane encapsulating a gas core, the lipid membrane including a lipid and a stabilizing agent;
wherein the compressible suspension has a low viscosity such that it is free of trapped gas.

19. The method of claim 18, wherein the compressible suspension is administered by intravenous or intraarterial injection.

20. The method of claim 18, wherein the compressible suspension is delivered by a system comprising:
a first container filled with a concentrated suspension comprising the gas-filled microbubbles at a concentration of at least 70% by volume,
a second container filled with an aqueous solution, and
a third container that has a first port connected to the first container, a second port connected to the second container, a third port for releasing trapped gas, and a fourth port connected to a drug delivery device,
wherein the concentrated suspension and the aqueous solution is mixed in the third container to form the compressible suspension with low viscosity.

21. The method of claim 20, wherein the system further comprises a first pump that controls flow of the suspension from the first container to the third container and a second pump that controls flow of the aqueous solution from the second container to the third container.

22-23. (canceled)

24. The method of claim 18, wherein the compressible suspension is delivered by a system comprising:
an inner bag filled with the compressible suspension, the inner bag including a port connected to a drug delivery device, and
an outer bag surrounding the inner bag,
wherein the system is configured such that filling a solution into the space between the inner bag and the outer bag results in flow of the compressible suspension from the inner bag to the drug delivery device.

25-26. (canceled)

27. The method of claim 18, wherein the compressible suspension is delivered by a system comprising at least one drug delivery device for housing the compressible suspension, wherein the drug delivery device has a first port connected to a tube, the first port having a diameter sufficient to release the compressible suspension into the tube at a flow rate of at least 10 mL/minute, wherein the drug delivery device has a second port for releasing trapped gas, and wherein the drug delivery device has a pressure unit for applying pressure to the compressible suspension to cause the compressible suspension to exit through the first port at the flow rate of at least 10 mL/minute.

28-32. (canceled)

33. The method of claim 32, wherein oxygen is delivered at an infusion rate of 10 to 400 ml/minute to the subject.

34. The method of claim 32, wherein the subject is or is suspected of experiencing local or systemic hypoxia.

35-44. (canceled)

45. A method for delivering a gas into a subject, the method comprising administering to a subject in need thereof an effective amount of a compressible suspension containing gas-filled microbubbles, each of which includes a lipid membrane encapsulating a gas core, wherein the lipid membrane contains (a) 1,2-disteroyl-sn-glycero-3-phosphocholine (DSPC) or dipalmitoylphosphatidylcholine (DPPC), and (b) one or more stabilizing detergents selected from the group consisting of poloxamer 188, a poloxamer having a molecular weight lower than that of poloxamer 188, Pluronic F108, Pluronic F127, polyoxyethylene (100) stearyl ether, cholesterol, gelatin, polyvinylpyrrolidone (PVP), and sodium deoxycholate (NaDoc).

46. The method of claim 45, wherein the gas core consists of oxygen, carbon dioxide, carbon monoxide, nitric oxide, inhalational anesthetic, hydrogen sulfide, or a mixture thereof.

47. The method of claim 45, wherein the compressible suspension has a low viscosity such that it is free of trapped gas.

48-89. (canceled)

90. A method for organ preservation, comprising delivering an effective amount of a suspension containing oxygen-filled microbubbles into a blood vessel in an organ, wherein each of the microbubbles contains a lipid membrane encapsulating a gas core that contains oxygen, the lipid membrane including a lipid and a stabilizing agent.

91-93. (canceled)

94. A method for prolonging storage of blood in vitro, comprising mixing oxygen-filled microbubbles with a blood sample, wherein the microbubbles each contain a lipid membrane encapsulating a gas core that contains oxygen and the lipid membrane includes a lipid and a stabilizing agent.

95. A method for promoting wound healing, comprising administering an effective amount of a suspension containing oxygen-filled microbubbles to a wound site or a site nearby a wound, wherein the microbubbles each contain a lipid membrane encapsulating a gas core that contains oxygen and the lipid membrane includes a lipid and a stabilizing agent.

96. (canceled)

97. A composition, comprising gas-filled microbubbles each of which contains a lipid membrane encapsulating a gas core, the lipid membrane including a lipid and a stabilizing agent, wherein the composition is formulated for topical administration.

98-99. (canceled)

100. A method for improving efficacy of a cancer radio therapy or reducing damage to non-cancerous tissues caused by the radio therapy, or for ameliorating sickle cell crisis, comprising administering an effective amount of a suspension containing oxygen-filled microbubbles to a tumor site or a site nearby a tumor in a subject who has undergone radio therapy, wherein the microbubbles each contain a lipid membrane encapsulating a gas core that contains oxygen and the lipid membrane includes a lipid and a stabilizing agent.

101-104. (canceled)

105. An non-invasive method for determining cardiac output, the method comprising
injecting a predetermined amount of oxygen into the venous bloodstream of a subject in need thereof,
measuring a time period needed for a change in expired oxygen content or a change in arterial oxygen saturations to occur, and
determining whether the subject's cardiac output based on the time period.

106. A system for delivering gas into a subject, the system comprising
a first container filled with a concentrated suspension comprising gas-filled microbubbles at a concentration of at least 70% by volume, wherein each of the microbubbles contains a gas core encapsulated by a lipid membrane that includes a lipid and a stabilizing agent,
a second container filled with an aqueous solution, and
a third container that has a first port connected to the first container, a second port connected to the second container, a third port for releasing trapped gas, and a fourth port for connecting to a drug delivery device.

107. (canceled)

108. A system for delivering gas into a subject, the system comprising
an inner bag filled with a suspension comprising gas-filled microbubbles, each of which contains a gas core encapsulated by a lipid membrane that includes a lipid and a stabilizing agent suspension, the inner bag having a port for connecting to a drug delivery device, and
an outer bag surrounding the inner bag,
wherein the system is configured such that filling a solution into the space between the inner bag and the outer bag results in flow of the suspension out of the inner bag through the port.

109. A system for administering a compressible suspension that comprises gas-filled microbubbles to a subject, comprising at least one drug delivery device for housing the compressible suspension, wherein the drug delivery device has a first port connected to a tube, the first port having a diameter sufficient to release the compressible suspension into the tube at a flow rate of at least 10 mL/minute, wherein the drug delivery device has a second port for releasing trapped gas, and wherein the drug delivery device has a pressure unit for applying pressure to the compressible suspension to cause the compressible suspension to exit through the first port at the flow rate of at least 10 mL/minute.

110-114. (canceled)

115. A syringe-based infusion apparatus, comprising:
a first chamber at a first end of the apparatus for housing gas or gas-filled microbubbles,
a second chamber at a second end of the apparatus for housing an aqueous diluent, a filter plate separating the first chamber and the second chamber, the filter plate including one central hole and a plurality of peripheral holes, in each of which a filter resides,
a plunger shaft attached to a compressing disc and a plunger disc, the plunger shaft, the compressing disc, and the plunger disc being movable along the axis of the apparatus integrally, and,
a port at the first end of the apparatus for connecting the first chamber to a delivery device, wherein the plunger shaft, the compressing disc, and the plunger disc are configured such that movement of the plunger shaft from the first end toward the second end of the apparatus causes movement of the compressing disc inside the second chamber toward the filter plate, forcing the aqueous diluent to flow from the second chamber into the first chamber.

116-134. (canceled)
